(19) United States
(12) Patent Application Publication
(10) Pub. No.: US 2024/0293345 A1
(43) Pub. Date: Sep. 5, 2024

(54) COMBINATIONS OF LOCAL ANESTHETICS FOR IMPROVED PAIN RELIEF

(71) Applicant: Pharmavax AS, Trollåsen (NO)

(72) Inventors: Morten Paulsbo, Vinterbro (NO); Magnus Paulsbo, Oppegård (NO)

(73) Assignee: Pharmavax AS, Trollåsen (NO)

(21) Appl. No.: 18/569,813

(22) PCT Filed: Jun. 14, 2022
(86) PCT No.: PCT/EP2022/066235

§ 371 Date: Dec. 13, 2023

(30) Foreign Application Priority Data
Jun. 14, 2021 (EP) 21179336.9

(51) Int. Cl. A61K 31/167; A61K 31/137; A61K 31/245; A61K 38/095; A61P 23/02

(52) U.S. Cl. CPC A61K 31/167; A61K 31/137; A61K 31/245; A61K 38/095; A61P 23/02

(57) ABSTRACT

The present invention relates to combinations of local anesthetics for improved pain relief in patients.

### FIELD OF THE INVENTION

The present invention is related to use of combinations of local anesthetics for improved anesthetic effect for patients which has or has had natural red hair or natural dark hair, especially patients with a mutation in the melanocortin 1 receptor (MC1R) gene.

### BACKGROUND

Local anesthetics is used to induce absence of sensation in a specific part of the body and are useful for a number of situations such as surgical procedures and dental procedures. Both topical and injectable formulations are well known.

Effective management of pain is essential to carry out surgical or dental procedures. Typical examples of local anesthetics are ester types such as benzocaine, novocaine, tetracaine, and amide types such as bupivacaine, lidocaine, ropivacaine and prilocaine.

Studies have shown that local anesthesia fails in 10% of cases of inferior alveolar nerve block and 7% of all cases of local anesthesia in general practice. Inferior alveolar nerve block is a procedure which is commonly used for local anesthesia in relation to dental procedures. Rev Belge Med Dent (1984) PMID: 11039282

Fear of pain (algophobia) is often rooted in previous experiences where a patient has had inadequate pain relief when in need thereof. Algophobia may cause avoidance behavior where a patient fails to seek help for an underlaying condition. This may lead to a worsening of said condition.

Dental care-related anxiety (odontophobia) is a problem which many suffer from. It is typically related to fear of dental pain and often results in avoidance of dental care, which in turn leads to further dental problems. Fear of dental pain is often rooted in previous bad experiences related to dental care due to inadequate pain relief

It is known that people with red hair are more likely to suffer from odontophobia. Red hair is related to a mutation in the melanocortin 1 receptor (MC1R) in humans. The human MC1R is expressed on the surface of melanocytes and is a key regulator of intracellular signaling to the melanin biosynthetic pathway governing pigment formation. In general, the balance of pheomelanin (yellow-red) and eumelanin (dark brown) pigments determines hair and skin color in the Caucasian population.

A mutation in MC1R cause excess of pheomelanin that result in phenotype red hair. The red hair phenotype is recessive, both parents need to pass along a recessive genetic trait for their child to have red hair. If a person inherits a MC1R mutation from only one parent, they will typically not have phenotype red hair, but rather have other color hair, such as blonde, brown or dark. They inherit mutations in the melanocortin 1 receptor on chromosome 16.

Binkley C. J. et al J AM Dent Assoc. 2009 July; 140(7): 896-905. doi: 10.14219/jada.archive.2009.0283. “Genetic variations associated with red hair color and fear of dental pain, anxiety regarding dental care and avoidance of dental care”

It has been established that redheads show increased sensitivity to pain and that they are resistant to the anesthetic effects of local anesthetics such as novocaine and lidocaine given subcutaneously.

MC1R is responsible not only for hair and skin color, but also for the midbrain function that determines pain response. Carriers of the MC1R mutation without natural red hair, i.e. other hair colors such as blonde, brown or dark hair will also experience insufficient local anesthesia.

Liem. E. B. et al; Anesthesiology. 2005 March; 102(3): 509-514. “Increased Sensitivity to Thermal Pain and Reduced Subcutaneous Lidocaine Efficacy in Redheads”

In U.S. Pat. No. 6,075,059A efforts in regard of providing local anesthetics with better efficacy has been made. A pharmaceutical composition comprising water, one local anesthetic such as lidocaine and a sugar alcohol at a concentration of at least 0.1 M was provided to enhance local anesthesia in the mouth region. This document does not address MC1R mutations and reduced effect of local anesthetics.

Cook et al. performed a study on the use of a combination of a first cartridge of 4% prilocaine plain plus a second cartridge of 2% lidocaine with 1:100,000 epinephrine or a combination of 2 cartridges of 2% lidocaine with 1:100,000 epinephrine. The two combinations were randomly given at two separate appointments to 118 subjects. In this study the goal was to compare the pain associated with the deposition of the respective anesthetic solutions and the degree of pulpal anesthesia obtained when used for inferior alveolar nerve block. The theory was that prilocaine may induce less pain upon administration due to having a high pH. Pain associated with anesthetic solution deposition from the first cartridge of 4% prilocaine plain was significantly less when compared with the first cartridge of 2% lidocaine with 1:100,000 epinephrine. There was not found any significant difference in pulpal anesthetic success between the two combinations.

Anesthetic Efficacy of a Combination of 4% Prilocaine/2% Lidocaine with Epinephrine for the Inferior Alveolar Nerve Block: A Prospective, Randomized, Double-blind Study, Cook et. al. 2018

DOI: https://doi.org/10.1016/j.joen.2018.01.004

None of the above cited documents shows that efforts have been made to target MC1R mutants and providing them with more effective local anesthesia.

It is therefore an object of the present invention to provide a pharmaceutical dosage form for use in effective local anesthesia for patients with MC1R-mutations.

Patients that are subjected to negative experiences, such as inadequate pain relief, in treatment situations where local anesthetics are used are at risk of developing algophobia and odontophobia.

It is therefore also an object of the present invention to provide a pharmaceutical composition for use in prophylaxis of algophobia and odontophobia.

### SUMMARY OF THE INVENTION

The experiments and disclosures provided herein are based on 9 case studies performed on patients that had, or previously has had phenotype red hair. The inventors performed odontological procedures on the patients and compared pain relief to prior experiences with local anesthetics.

The inventors have surprisingly found that administration of a combinations of a first local anesthetic of amino amide-type or amino ester-type, that is not prilocaine, with a second local anesthetic that is prilocaine is particularly advantageous and effective as a local anesthetic in patients with MC1R mutations, and thus also particularly advantageous in use for prophylaxis of algophobia and odontophobia.

Thus, a first aspect of the present invention relates to a pharmaceutical composition for use in prophylaxis in a patient, of at least one condition selected from a group consisting of algophobia and odontophobia, wherein the pharmaceutical composition comprises a combination of a first local anesthetic and a second local anesthetic, wherein the first local anesthetic is selected from a group consisting of amino amide-local anesthetics and amino ester-local anesthetics and pharmaceutically acceptable salts thereof, and the second local anesthetic is prilocaine or a pharmaceutically acceptable salt thereof, and optionally further pharmaceutically acceptable excipients,

and wherein the first local anesthetic is not prilocaine.

A second aspect of the present invention relates to a pharmaceutical composition for use in treatment of pain in a patient, wherein the pharmaceutical composition comprises a combination of a first local anesthetic and a second local anesthetic, wherein the first local anesthetic is selected from the group consisting of amino amide-local anesthetics and amino ester-local anesthetics and pharmaceutically acceptable salts thereof, and the second local anesthetic is prilocaine or a pharmaceutically acceptable salt thereof, and optionally further pharmaceutically acceptable excipients,

and wherein the first local anesthetic is not prilocaine.

In one example of the pharmaceutical composition for use according to the first or second aspect, the pharmaceutical composition is for injection.

In one example of the pharmaceutical composition for use according to the first or second aspect, the first local anesthetic is selected from a group consisting of butacaine, benzocaine, chloroprocaine, hexylcaine, piperocaine, procaine propoxycaine, tetracaine, articaine, bupivacaine, dibucaine, etidocaine, lidocaine, mepivacaine or ropivacaine and pharmaceutically acceptable salts thereof.

In one example of the pharmaceutical composition for use according to the first or second aspect, the first local anesthetic is lidocaine or a pharmaceutically acceptable salt thereof.

In one example of the pharmaceutical composition for use according to the first or second aspect the pharmaceutical composition comprises a vasoconstrictor.

In one example the pharmaceutical composition for use according to the first or second aspect the vasoconstrictor is selected from a group consisting of epinephrine and felypressin.

In one example the pharmaceutical composition for use according to the first or second aspect the patient previously has had inadequate local anesthesia, when in need thereof, when receiving one local anesthetic selected from the group consisting of amino amides and amino esters and pharmaceutically acceptable salts thereof.

In one example of the pharmaceutical composition for use according to the first or second aspect the patient has at least one mutation in at least one allele of a melanocortin 1 receptor (MC1R) gene, wherein the at least one mutation is a loss-of-function mutation.

In one example the pharmaceutical composition for use according to the first or second aspect the patient has phenotype red hair.

In one example the pharmaceutical composition for use according to the first or second aspect the first local anesthetic and the second local anesthetic are comprised in the same container.

In one example the pharmaceutical composition for use according to the first or second aspect the first local anesthetic and the second local anesthetic are comprised in the same container and in solution together.

In one example the pharmaceutical composition for use according to the first or second aspect the first local anesthetic and the second local anesthetic are comprised in separate containers.

In one example the pharmaceutical composition for use according to the first or second aspect the pharmaceutical composition is for injection in the oral cavity.

In one example the pharmaceutical composition for use according to the first or second aspect the pharmaceutical composition is for injection in the mouth region.

In a third aspect the present invention relates to a pharmaceutical composition for injection for use in a method for prophylaxis in a patient of at least one condition selected from a group consisting of algophobia and odontophobia, wherein the pharmaceutical composition comprises a combination of a first local anesthetic and a second local anesthetic, wherein the first local anesthetic is selected from a group consisting of amino amide-local anesthetics and amino ester-anesthetics and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine, and wherein the first local anesthetic and the second local anesthetic are comprised in separate containers, and wherein the first local anesthetic is administered before, simultaneously or after the second local anesthetic.

In another aspect the present invention relates to a pharmaceutical composition for injection for use in a method for treatment of pain in a patient, wherein the pharmaceutical composition comprises a combination of a first local anesthetic and a second local anesthetic, wherein the first local anesthetic is selected from a group consisting of amino amide-local anesthetics and amino ester-local anesthetics and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine, and wherein the first local anesthetic and the second local anesthetic are comprised in separate containers, and wherein the first local anesthetic is administered before, simultaneously or after the second local anesthetic.

In one example of the pharmaceutical composition for use in a method the first local anesthetic is lidocaine or a pharmaceutically acceptable salt thereof.

In one example of the pharmaceutical composition for use in a method the first local anesthetic is administered before the second local anesthetic.

In one example of the pharmaceutical composition for use in a method the patient previously has had inadequate local anesthesia, when in need thereof, when receiving one local anesthetic selected from the group consisting of amino amides and amino esters and pharmaceutically acceptable salts thereof.

In one example of the pharmaceutical composition for use in a method patient has at least one mutation in at least one allele of a MC1R gene, wherein the at least one mutation is a loss-of-function mutation.

In one aspect the present invention relates to a pharmaceutical composition for injection comprising a combination of a first local anesthetic and a second local anesthetic, wherein the first local anesthetic is selected from a group consisting of amino amide-local anestheticss and amino ester-local anestheticss and pharmaceutically acceptable salts thereof, and the second local anesthetic is prilocaine or pharmaceutically acceptable salts thereof, and a vasoconstrictor, and optionally further pharmaceutically acceptable excipients, and wherein the first local anesthetic is not prilocaine.

In one example of the pharmaceutical composition for injection the first local anesthetic is selected from the group consisting of butacaine, benzocaine, chloroprocaine, hexylcaine, piperocaine, procaine propoxycaine, tetracaine, articaine, bupivacaine, dibucaine, etidocaine, lidocaine, mepivacaine or ropivacaine and pharmaceutically acceptable salts thereof.

In one example of the pharmaceutical composition for injection the first local anesthetic is lidocaine or a pharmaceutically acceptable salt thereof.

In one example of the pharmaceutical composition for injection comprises a vasoconstrictor.

In one example of the pharmaceutical composition for injection the vasoconstrictor is epinephrine or felypressin.

In one example of the pharmaceutical composition for injection the first local anesthetic is lidocaine or a pharmaceutically acceptable salt thereof, the second local anesthetic is prilocaine or a pharmaceutically acceptable salt thereof

In one example of the pharmaceutical composition for injection the first local anesthetic is lidocaine or a pharmaceutically acceptable salt thereof, the second local anesthetic is prilocaine or a pharmaceutically acceptable salt thereof and the vasoconstrictor is epinephrine and/or felypressin.

In one example the pharmaceutical composition is for injection, wherein the injection is provided to the mouth region or the oral cavity.

In one example the pharmaceutical composition is for injection, wherein the first local anesthetic and second local anesthetic is for administration simultaneously.

It is understood that all exemplary embodiments may apply to the different aspects of the present invention.

### Definitions

As used herein the term “container” is used in the meaning of any container suitable for holding a pharmaceutical composition for injection, such a vial or a cartridge.

As used herein the term “pharmaceutical injection composition” is to be understood as a composition that is for injection use to a patient.

As used herein the term “loss-of-function mutation”, also called inactivating mutations, is mutations resulting in the gene product having less or no function i.e. being partially or wholly inactivated.

As used herein the term “treatment of pain” is understood as alleviating pain, numbing of a body part, relief of pain, ease unpleasant sensation or provide anesthesia, treatment in order to help a patient or person to cope with unpleasant feelings, the treatment may also be to avoid pain caused by a practitioner performing surgical procedures, dental procedures or other unpleasant procedures.

As used herein the term “prophylaxis” is understood as measures to preserve health, actions taken to prevent certain conditions or problems such as phobias, measures to be taken in advance to avoid certain problems.

### DETAILED DESCRIPTION OF THE INVENTION

Unless specifically defined herein, all technical and scientific terms used have the same meaning as commonly understood by a skilled artisan in the fields of genetics, biochemistry, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail.

Where a numeric limit or range is stated, the endpoints are included. Also, all values and subs ranges within a numerical limit or range are specifically included as if explicitly written out.

As discussed above patients with MC1R-mutations show increased sensitivity to pain and that the same patient group is resistant to the effects of local anesthetics, such as novocaine and lidocaine when given subcutaneously. MC1R-mutations are recessive. If a subject has a MC1R-mutation in both alleles they will have the phenotype red hair, which is a clear marker of MC1R-mutations. However, if a subject only inherits the mutation in one allele, the subject may be of phenotype blonde hair, brown hair, dark hair, but will still experience inadequate effects of local anesthetics. Given that MC1R-mutations appear in subjects of all hair colors, one cannot easily predict which patient will experience inadequate pain relief.

Currently the solution for treatment of non-responsive patients have been to administer larger doses of local anesthetics than for patients that respond normally to local anesthetics. This practice has not been successful in providing effective pain relief in many cases and may also lead to unwanted side effects due to the larger doses that non-responsive patients may receive.

Patients that experience inadequate pain relief in treatment situations may develop algophobia and/or odontophobia.

There is therefore a need for an effective local anesthetic for patients with MC1R mutations. Also, there is a need for prophylactic treatment of inadequate pain relief, algophobia and odontophobia.

The present inventors have surprisingly found that administering a combination of lidocaine and prilocaine has advantageous and desirable effects on local anesthesia in patients with MC1R mutations.

Local anesthetics includes drugs of ester type such as benzocaine, novocaine, tetracaine, and amide types such as bupivacaine, lidocaine, ropivacaine and prilocaine. These nonlimiting examples are used to in context of surgery, dental procedures and other interventions on a mammal, such as a human, when absence of pain sensation is desirable. A local anesthetic creates an absence of pain sensation at the point of administration on the body. This is useful when it is desired to keep a patient awake during a procedure as opposed to a general anesthetic which often causes loss of consciousness. When used on specific nerve pathways, a local anesthetic can be used to cause nerve block which result in loss of pain sensation. One example of this is alveolar nerve block, which is typically obtained by injection of a local anesthetic into the region of the alveolar nerve. This is a particularly common practice when a patient is to receive dental treatment which causes pain, such as drilling, extractions, crown work, root canal work, etc.

The mechanism of action for the different local anesthetics are similar. The ionised form of local anesthetics bind to open voltage-gated Na+ channels in a reversible and concentration-dependent manner. The binding site for local anaesthetics is located in domain IV, loop S6 and is only accessible when the channel is open. The binding of local anaesthetics to open Na+ channels increases with the frequency of nerve depolarisation. Bound local anaesthetic drug stabilises the inactivated receptor state, preventing further neuronal transmission. Local anaesthetic nerve block is concentration-dependent. With increased concentrations of local anaesthetic, the peak of the action potential is reduced, the firing threshold increases, impulse conduction is attenuated, and the refractory period lengthened. Increased concentrations inhibit all nerve conduction.

Two commonly used local anesthetics are lidocaine and prilocaine.

Lidocaine is also known as lignocaine and is sold under the brand name Xylocaine. Lidocaine is a local anesthetic of the amino amide type and has the following structure:

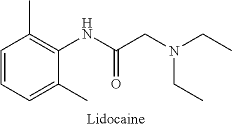 [SMILES: CCN(CC)CC(=O)Nc1c(C)cccc1C]

Lidocaine is assigned the CAS registry number 137-58-6. When used for local anesthesia or in nerve blocks, lidocaine typically shows effect within several minutes and last for 0.5 h-3 h. Lidocaine administration routes include injections, or topical to mucosa or directly to skin to obtain an absence of pain sensation.

In order to prolong effects, to keep the effects local or to prevent or decrease bleeding lidocaine is often mixed and administered epinephrine. Epinephrine will then act as a vasoconstrictor via agonist action on al receptors. The vasoconstriction helps to keep the lidocaine at the place of administration due to decreased vascular flow.

Lidocaine alters signal conduction in neurons by binding to and prolonging the inactivation of the fast voltage-gated Na+ channels in the neuronal cell membrane responsible for action potential propagation. With sufficient blockage, the voltage-gated sodium channels will not open and an action potential will not be generated. Careful titration allows for a high degree of selectivity in the blockage of sensory neurons, whereas higher concentrations also affect other types of neurons. It is understood that higher doses can lead to higher risk of side effects.

Lidocaine for injection is available for example in vails of 20 mL in concentrations of 10 mg/mL and 20 mg/mL.

Lidocaine with epinephrine for injection is available for example as cartridges of 1.8 mL comprising lidocaine 20 mg/mL and epinephrine 12.5 μg/mL, also denoted a 2% concentration. Other concentrations are also available such as 0.5% and 1%. Other concentrations of epinephrine are also available, such as 5 μg/mL and 10 μg/mL.

The concentration of epinephrine may be 3 μg/mL-30 μg/mL, 5 μg/mL-20 μg/mL, 10 μg/mL-20 μg/mL.

The concentration of lidocaine may be from 0.1%-10%, 0.2%-5%, 0.3%-3%.

The total dose of lidocaine varies depending on the type of nerve-blockade that is wanted. The dosage of lidocaine is typically lower for local anesthetic effect where blockade of single nerves is needed, and the dosage of lidocaine may be larger when anesthetic effect for larger areas are needed. The total dosage may be from 5-1000 mg, 10-700 mg, 15-500 mg, 20-400 mg, 30-300 mg, 40-200 mg, 50-150 mg, 60-100 mg, 70-90 mg or 80 mg.

It is to be understood that the present invention allows for use of lidocaine with or without epinephrine for all embodiments described herein.

Prilocaine is a local anesthetic of the amino amide type and is sold under the brand name Citanest which comprises prilocaine and felypressin. Prilocaine has the following structure:

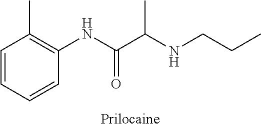 [SMILES: CCCNC(C)C(=O)Nc1ccccc1C]

Prilocaine is assigned the CAS registry number 721-50-6. The injectable form of prilocaine is often used in dentistry. Prilocaine has a clinical profile similar to lidocaine and is used for infiltration, peripheral nerve blocks, and spinal and epidural anesthesia. Prilocaine metabolism is similar to that of lidocaine, being rapidly metabolized by the liver and excreted in the urine as o-toluidine.

Prilocaine for injection is available for example in vails of 5 mL in concentrations of 20 mg/mL. Other concentrations may also be used, such as 5 mg/mL or 10 mg/mL.

Felypressin, a synthetic analogue of vasopressin, is sometimes used in combination with prilocaine as a vasoconstrictor to reduce absorption of and localize the effect of prilocaine.

Prilocaine with felypressin for injection is available for example in cartridges of 1.8 mL comprising prilocaine HCL 30 mg/mL and felypressin 0.54 μg/mL also denoted as 3% concentration. Other concentrations such as 4% and 2% may also be used for the same purpose.

The concentration of felypressin may be 0.2 μg/mL-5 μg/mL, 0.3 μg/mL-4 μg/mL, 0.4 μg/mL-2 μg/mL.

The concentration of prilocaine may be from 0.1%-10%, 0.2%-5%, 0.5%-3%.

The total dose of prilocaine varies depending on the type of nerve-blockade that is wanted. The dosage of prilocaine is typically lower for local anesthetic effect where blockade of single nerves is needed, and the dosage of lidocaine may be larger when anesthetic effect for larger areas are needed. The total dosage may be from 5-1000 mg, 10-700 mg, 15-500 mg, 20-400 mg, 30-300 mg, 40-200 mg, 50-150 mg, 60-100 mg, 70-90 mg or 80 mg.

The inventors have administered lidocaine and prilocaine by injection.

Genetic Variations Associated with Red Hair.

Red hair nearly always results from mutations in the melanocortin-1 receptor gene (MC1R). The MC1R is expressed on the surface of melanocytes and is a key regulator of pigment formation. The red hair phenotype results from excess pheomelanin production due to dysfunctional MC1Rs. Loss-of-function mutations at the MC1R are associated with this switch from eumelanin to phacomelanin production. In contrast, normal MC1R expression results in a dominant production of eumelanin pigments which are dark brown. The red hair phenotype is recessive and is only displayed if both alleles show the mutation. Thus, it cannot be determined that a patient does not have a MC1R mutation based on that the patient does not have the red hair phenotype. Patients with brown, black and blonde hair may also have a MC1R mutation.

The MC1R gene is located on chromosome 16 (16924.3) and comprises a single 951 bp long exon. The gene encodes the typical G protein-coupled receptor associated with the melanocyte membrane, and its activation increases the CAMP level. Some alleles of the MC1R, described as diminished function alleles or alleles that affect the melanocyte membrane expression of the receptor, are significantly associated with the overproduction of pheomelanin, which manifests in red hair and fair skin. Variation within the MC1R gene is exceptionally high among Caucasians and has a significant impact on the pigmentary phenotype in this ethnic group.

One method known to the skilled person for identifying individuals with loss-of-function mutations of the MC1R gene is MC1R-genotyping as described in Kanetsky, P. A., et al: “Assessment of Polymorphic Variants in the Melanocortin-1 Receptor Gene with Cutaneous Pigmentation Using an Evolutionary Approach. Cancer Epidemiology Biomarkers 2004; 13(5)”, which is incorporated herein by reference.

MC1R Genotyping was performed in the following manner by Kanetsky et al: Genomic DNA was extracted from collected buccal swab samples. One of two PCR protocols was used to amplify the entire 951 nucleotide MC1R coding region. Briefly, the 50 Al total reaction volume contained 10 Al DNA template, 5 AM each forward 5V-GCAGCACCATGAACTAAGCA-3V and reverse 5V-CAGGGTCACACAGGAACCA-3V primers, 200 μl each dATP, dCTP, dGTP, dTTP, 5 μl DMSO, 5 μl 10× Herculase Taq polymerase buffer (containing MgCl2; Stratagene, La Jolla, CA), 1.0 μl (5 units) Herculase Taq polymerase (Stratagene), and ddH2O. Thermocycling occurred in a Robocycler 96 (Stratagene). DNA template was denatured at 94° C. for 3 min and cycled 35 times through steps of denaturing at 94° C. for 1 min, annealing at 55° C. for 1 min, and extension at 72° C. for 3 min; final DNA extension was at 72° C. for 7 min. The original protocol used three sets of overlapping oligonucleotide primer pairs to amplify MC1R and was replaced with the more efficient single primer protocol. Regardless of amplification protocol, all PCR products were directly sequenced on an ABI Prism 377 or 3100 (Applied Biosystems, Foster City, CA) using Big Dye Terminators (Applied Biosystems) according to the manufacturers specifications.

There has been found suggestions that MC1R is involved in pain modulation. MC1R expression has been identified in human pituitary tissue, glial cells, and in human periaqueductal grey matter. However, the central nervous system (CNS) is not a major site of MC1R expression and it is therefore not entirely clear why MC1R mutations should alter pain sensation and local anesthetic requirements.

Without being bound by theory, a possible explanation is that MC1R mutations up-regulates production of the receptors primary ligands, the melanocortins, including α-melanocyte-stimulating hormone, which also stimulates other melanocortin receptors such as the melanocortin-4 receptors that modulates cold and mechanical allodynia in rat neuropathic models. (Liem, E B et al 2005, Anesthesiology 2005, 102:509-14)

However, there has been many reports of redheads that claims that local anesthesia either often fails or that unusually large doses of local anesthetics to achieve adequate local anesthesia.

Identification of patients that have previously experienced inadequate local anesthetic effect in relation to administration of one local anesthetic selected from the group consisting of amino amides and amino esters and pharmaceutically acceptable salts thereof the may for example be done by asking the patent to perform a rating of the pain experienced during the previous procedure performed under local anesthesia on a “pain scale” such a visual analogue scale (VAS), wherein a “pain scale” is a scale from 0-10 illustrating the subjective feeling of pain by a patient where 0 is no pain and 10 is the worst imaginable pain. Patients reporting pain may for example report a VAS value of 1-10, 2-10, 3-10 or 4-10.

There has also been found a correlation between the red hair phenotype and fear of dental pain and dental care in general. It is believed that this increased anxiety can lead to algophobia and/or odontophobia which in turn are known reasons for avoidance behavior. When patients start avoiding dental care, it does not take long before the need for dental care aggregates, possibly making the procedures needed more complex and painful.

The present inventors have surprisingly found that using a combination of lidocaine and prilocaine is particularly advantageous in the treatment of pain in patients with MC1R mutations. Said combination of lidocaine and prilocaine consistently resulted in superior local anesthesia compared to that of lidocaine alone when administered to said patients with MC1R mutations in need of local anesthesia.

The present inventors have surprisingly found that using a combination of lidocaine and prilocaine is particularly advantageous in treatment of patients that have developed algophobia and odontophobia due to previous inadequate pain relief from previous experiences with local anesthetics. This is especially true for patients with MC1R mutations, since they will have experience adequate pain relief when using the combination lidocaine and prilocaine. Patients that finally experience adequate pain relief will overcome their algophobia and odontophobia.

Identification of patients that suffer from algophobia and/or odontophobia can for example be done using a questionnaire providing questions that reveals whether a patient is suffering from algophobia and/or odontophobia.

The present inventors have surprisingly found that using a combination of lidocaine and prilocaine is particularly advantageous in prophylaxis of algophobia and odontophobia in the general population of patients, since use of said combination will keep patients with MC1R-mutations from experiencing that first episode of inadequate pain relief. This would be of help for patients with MC1R mutations with phenotype red hair and patients with other hair colors that might still have the MC1R mutation.

Not being bound by theory, the present inventors contemplates that the synergistic effects seen by administration of a combination of lidocaine and prilocaine is due to enhanced effects on the sodium channels responsible for pain sensation. Local anesthetics act mainly by inhibiting sodium influx through sodium specific ion channels in the neuronal cell membrane, in particular in the voltage-gated sodium channels. When influx of sodium is interrupted, an action potential cannot arise and signal conduction along the neuron is inhibited. The binding of lidocaine and prilocaine to the sodium channels seems to trigger the synergistic and beneficial effect for patients with MC1R mutations. Furthermore, it is contemplated that the beneficial effect observed is due to the local anesthetics.

As used herein the term “red hair” refers to the multiple tints, tones and shades of the color red. Mutations in the MC1R may give rise to many variations of the color red, including light red, bright red, dark red, maroon, scarlet, Indian red, tomato, orange-red, yellow-red, red, firebrick, crimson, light coral, merlot and many more.

The color of red hair resulting from MC1R mutations may be defined by Munsell's color system having a Red to Yellow red color and varying value of lightness and chroma. (Munsell color system)

In a first aspect the present invention provides a pharmaceutical composition for use in the prophylaxis of at least one condition selected from the group consisting of algophobia and odontophobia in a patient, wherein the pharmaceutical composition comprises a combination of a first local anesthetic and a second local anesthetic, wherein the first local anesthetic is selected from the group consisting of amino amides and amino esters and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine.

In general lidocaine is preferred as the first local anesthetic and prilocaine is preferred as the second local anesthetic, although the first local anesthetic may be other local anesthetics than lidocaine, preferably local anesthetics of the ester type or the amide type with similar properties to lidocaine.

The pharmaceutical composition for use according to the first aspect represents a novel means for prophylaxis of algophobia and odontophobia, helping patients that are at risk of developing avoidance behavior.

In a second aspect the present invention provides a pharmaceutical composition for use in the treatment of pain in a patient, wherein the pharmaceutical composition comprises a combination of a first local anesthetic and a second local anesthetic, wherein the first local anesthetic is selected from the group consisting of amino amides and amino esters and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine.

The pharmaceutical composition for use according to the second aspect represents an improved and more effective option for providing patient with local anesthesia. In particular for patients with MC1R mutations and patients with phenotype red hair.

In a third aspect the present invention provides a pharmaceutical composition for injection for use in a method for prophylaxis of at least one condition selected from the group consisting of algophobia and odontophobia in a patient, wherein the pharmaceutical composition comprises a combination of a first local anesthetic and a second local anesthetic, wherein the first local anesthetic is selected from the group consisting of amino amides and amino esters and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine, and wherein the first local anesthetic and the second local anesthetic is comprised in separate containers, and wherein the first local anesthetic is administered before, simultaneously or after the second local anesthetic.

In a fourth aspect the present invention provides a pharmaceutical composition for injection for use in a method for treatment of pain in a patient, wherein the pharmaceutical composition comprises a combination of a first local anesthetic and a second local anesthetic, wherein the first local anesthetic is selected from the group consisting of amino amides and amino esters and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine, and wherein the first local anesthetic and the second local anesthetic is comprised in separate containers, and wherein the first local anesthetic is administered before, simultaneously or after the second local anesthetic.

In a fifth aspect the present invention provides a pharmaceutical composition for injection comprising a combination of a first local anesthetic and a second local anesthetic, wherein the first local anesthetic is selected from the group consisting of amino amides and amino esters and pharmaceutically acceptable salts thereof, and the second local anesthetic is prilocaine and pharmaceutically acceptable salts thereof, and a vasoconstrictor, and optionally further pharmaceutically acceptable excipients, and wherein the first local anesthetic is not prilocaine.

In another aspect the present invention relates to a pharmaceutical composition for use in treatment in a patient, of at least one condition selected from a group consisting of algophobia and odontophobia, wherein the pharmaceutical composition comprises a combination of a first local anesthetic and a second local anesthetic, wherein the first local anesthetic is selected from a group consisting of amino amide-local anesthetics and amino ester-local anesthetics and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine.

In another aspect the present invention provides a pharmaceutical composition for use in the prophylaxis of at least one condition selected from the group consisting of algophobia and odontophobia in a patient, wherein the patient is susceptible for development of odontophobia and/or algophobia, wherein the pharmaceutical composition comprises a combination of a first local anesthetic and a second local anesthetic, wherein the first local anesthetic is selected from the group consisting of amino amides and amino esters and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine.

In one aspect the present invention concerns a method for prophylaxis of at least one condition selected from the group consisting of algophobia and odontophobia in a patient, by providing the patient with a pharmaceutical composition comprising a combination of a first local anesthetic and a second local anesthetic, wherein the first local anesthetic is selected from the group consisting of amino amides and amino esters and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine.

In one aspect the present invention concerns a method for treatment of pain in a patient, by providing the patient with a pharmaceutical composition comprising a combination of a first local anesthetic and a second local anesthetic, wherein the first local anesthetic is selected from the group consisting of amino amides and amino esters and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine.

In one aspect the present invention concerns a method for treatment of at least one condition selected from a group consisting of algophobia and odontophobia, by providing the patient with a pharmaceutical composition comprising a combination of a first local anesthetic and a second local anesthetic, wherein the first local anesthetic is selected from a group consisting of amino amide-local anesthetics and amino ester-local anesthetics and pharmaceutically acceptable salts thereof, and the second local anesthetic is prilocaine or a pharmaceutically acceptable salt thereof, and optionally further pharmaceutically acceptable excipients, and wherein the first local anesthetic is not prilocaine.

### Embodiments

In general for the aspects of the present invention, lidocaine is preferred as the first local anesthetic and prilocaine is preferred as the second local anesthetic, although the first local anesthetic may be other local anesthetics than lidocaine, preferably local anesthetics of the ester type or the amide type with similar properties to lidocaine.

In one example of the aspects of the present invention the first local anesthetic is not prilocaine, i.e. the first local anesthetic is a local anesthetic other than prilocaine.

In example of the aspects of the present invention the pharmaceutical composition further comprises a vasoconstrictor, such as epinephrine or felypressin for keeping the effect localized and prolonging the effect.

In example of the aspects of the present invention the pharmaceutical composition is administered to a subject by intravenous, intramuscular, transdermal, topical, sustained release, controlled release, delayed release, suppository, subcutaneous, catheter, topical, or sublingual administration.

In one example of the aspects of the present invention the pharmaceutical composition comprises the first local anesthetic and the second local anesthetic in one mixture.

In one example of the aspects of the present invention the pharmaceutical composition comprises the first local anesthetic and the second local anesthetic in separate mixtures in separate containers.

In one example of the aspects of the present invention the pharmaceutical composition comprises further pharmaceutically acceptable excipients such as solvents, pH-adjusting agents or surfactants.

In one example of the aspects of the present invention the pharmaceutical composition a preservative is needed in order to secure adequate shelf life of the product. Parabens may for example be suitable.

In one example of the aspects of the present invention the pharmaceutical composition is provided in cartridges. The volume of the cartridges may for example be 1-10 mL. 2-9 mL, 3-8 mL, 4-7 mL, 5-6 mL or 1.8 mL

In one example of the aspects of the present invention the pharmaceutical composition may have a concentration of lidocaine and/or prilocaine of 0.1-10%, 0.5-5%, 1-7%, w/w, 2-5% w/w, 3-4% w/w.

In one example of the aspects of the present invention the use is for alveolar nerve block.

In one example of the aspects of the present invention the pharmaceutical composition is for injection in the oral cavity.

In one example of the aspects of the present invention the pharmaceutical composition is for injection in the mouth region.

In one example the pharmaceutical composition for use according to the first or second aspect the pharmaceutical composition is for injection in the mouth region.

In one example of the aspects of the present invention the use is for peripheral anesthesia.

In one example of the aspects of the present invention the use is for peripheral nerve block, including brachial, intercostal, paravertebral and/or pudendal.

In another example of the aspects of the present invention the use is for percutaneous infiltration, intravenous regional infiltration, paracervical obstetrical analgesia, sympathetic nerve blocks such as cervical and lumbar, central nerve blocks such as epidural, thoracic and surgical anesthesia.

In one example of the aspects of the present invention the use of the pharmaceutical composition is for local anesthesia in other part of the body than the oral cavity. For example, the skin, joints, bones or muscles of a subject. If vasoconstriction is not important for keeping the effect local, the pharmaceutical composition does not include a vasoconstrictor.

The inventors have found that the particularly advantageous effects of the present invention is observed when the first local anesthetic is a local anesthetic other than prilocaine, and the second local anesthetic is prilocaine.

The inventors have in particular found that the advantageous effects of the present invention is observed when the first local anesthetic is lidocaine and the second local anesthetic is prilocaine.

The above-mentioned examples are applicable to all described aspects of the present invention.

### EXAMPLES

### Method:

9 patients with a history of poor pain relief after administration of just one local anesthetic received a combination of lidocaine and prilocaine before various dental procedures where performed. The patients pain sensation was the noted in case studies kept at the inventor's clinic.

It is noted that the inventors have previously observed cases where prilocaine alone as well as lidocaine alone did not provide adequate anesthetic effect on patients.

Commercially available Xylocain Dental Adrenaline (lidocaine HCL 20 mg/mL with epinephrine 12.5 μg/mL) and Citanest Dental Octopressin (prilocaine HCL 30 mg/mL and felypressin 0.54 μg/mL) was administered by injection to mouth region of the patient for pain relief. Both Xylocain Dental Adrenaline and Citanest Dental Octopressin was provided in cartridges containing 1.8 mL of solution comprising active ingredients in said concentrations.

### Example 1

Patient 24154: Female, born 1974.
Phenotype: Red hair.
Pronounced odontophobia

Experienced unsatisfactory effect of local anesthesia as a child, and into adulthood. The first time she had a good effect of local anesthesia was at the inventor's clinic.

Combination administered:
½ cartridge lidocaine with epinephrine.
½ cartridge prilocaine with felypressin.

Several odontological procedures had been done previously where one cartridge lidocaine with epinephrine was administered followed by administration of a second cartridge of lidocaine with epinephrine. Satisfactory effect was only obtained when administering the combination of lidocaine and prilocaine.

### Example 2

Patient 24248: Female, born 1957.
Phenotype: Previously brown hair, now grey hair.
Pronounced odontophobia.

Experienced unsatisfactory effect of local anesthesia as a child, and into adulthood. The first time she had a good effect of local anesthesia was at the inventor's clinic.

The patient had 2 treatments on the same day.

At the 1^{st} treatment the following was administered for local anesthesia:

### Combination Administered:

½ cartridge lidocaine with epinephrine.
½ cartridge prilocaine with felypressin.

Effective pain relief is obtained and 2 teeth are crowned without pain.

For the 2^{nd} treatment 1 cartridge of lidocaine with epinephrine was administered and an icing feeling was experienced upon blowing air onto the teeth. After administration of ½ cartridge of prilocaine with felypressin effective pain relief was obtained.

### Example 3

Patient 20314: Male, born 1953.
Phenotype: Brown hair.

Experienced unsatisfactory effect of local anesthesia as a child, and into adulthood. The first time he had a good effect of local anesthesia was at the inventor's clinic. The patient reported that it was very pleasant to experience effective pain relief.

### Combination Administered:

½ cartridge lidocaine with epinephrine.
½ cartridge prilocaine with felypressin.

### Example 4

Patient 24457: Male, born 1946.
Phenotype: Reddish hair.

Pronounced odontophobia. Severe anxiety for dental treatment, as he had never experienced effective local anesthesia and furthermore is very sensitive to pain. Pronounced need for treatment but was extremely anxious when entering the clinic.

### Combination Administered:

½ cartridge lidocaine with epinephrine.
½ cartridge prilocaine with felypressin.

Excellent pain relief obtained. Root canal treatment, extractions and filing therapy performed without pain over many treatment sessions.

Subsequent attempts with administration of lidocaine with epinephrine alone has not yielded successful pain relief.

### Example 5

Patient 192293: Male, born 1960.
Phenotype: Previously brown, now grey.

No odontophobia but has previously experienced inadequate effect of local anesthesia when drilling in teeth. Has always felt “some pain” despite administration of large doses of local anesthesia.

### Combination Administered:

½ cartridge lidocaine with epinephrine.
½ cartridge prilocaine with felypressin

Drilling in teeth and surgery with dental implant treatment was possible without pain.

The patient also describes an incident where he dislocated a shoulder. Three attempts of administration of lidocaine was performed without obtaining adequate pain relief. The shoulder was dislocated for a further 3 days before the patient was put in narcosis and operated. The patient needed further operations as a result of complications that might have been avoided if a combination of lidocaine and prilocaine had been administered at the first attempt to treat the dislocated shoulder.

### Example 6

Patient 120512: Female, born 1969.
Phenotype: Red hair.

Pronounced odontophobia as a result of inadequate effect of local anesthesia. Dental treatment was performed under narcosis but later treatment sessions were performed with local anesthesia with administration of a combination of lidocaine and prilocaine.

### Example 7

Patient 21484: Male, born 1957.
Phenotype: Red hair.

Mild odontophobia. Previous experiences with local anesthesia has been inadequate. Upon administration of a combination of lidocaine and prilocaine god pain relief is obtained. Lidocaine alone was not effective.

### Example 8

Patient 20388: Female, born 1975.
Phenotype: Red hair.

Poor effect obtained with lidocaine alone.

Administration of a combination of lidocaine and prilocaine yields effective pain relief.

### Example 9

Patient 23371: Male, born 1992.
Phenotype: Red hair.

The patient had aggregated need for dental treatment and showed avoidance behavior towards dental treatment. Had throughout his lifetime experienced poor effect of local anesthesia.

Lidocaine with epinephrine was first administered to the patient with poor effect. When administering a dose of prilocaine with felypressin effective local anesthesia is obtained.

1. A pharmaceutical composition for use in treatment of pain or prophylaxis of algophobia or odontophobia in a patient, wherein the pharmaceutical composition comprises
a combination of a first local anesthetic and a second local anesthetic, wherein
the first local anesthetic is selected from the group consisting of amino amide-local anesthetics, and amino ester-local anesthetics, and pharmaceutically acceptable salts thereof, and
the second local anesthetic is prilocaine or a pharmaceutically acceptable salt thereof, and
optionally pharmaceutically acceptable excipients,
and wherein the first local anesthetic is not prilocaine.

2. (canceled)

3. The pharmaceutical composition according to claim 1, wherein the pharmaceutical composition is for injection.

4. The pharmaceutical composition according to claim 1, wherein the first local anesthetic is selected from a group consisting of butacaine, benzocaine, chloroprocaine, hexylcaine, piperocaine, procaine propoxycaine, tetracaine, articaine, bupivacaine, dibucaine, etidocaine, lidocaine, mepivacaine or ropivacaine and pharmaceutically acceptable salts thereof.

5. The pharmaceutical composition according to claim 4, wherein the first local anesthetic is lidocaine or a pharmaceutically acceptable salt thereof.

6. The pharmaceutical composition according to claim 1, wherein the dosage form further comprises a vasoconstrictor.

7. The pharmaceutical composition according to claim 6, wherein the vasoconstrictor is selected from a group consisting of epinephrine and felypressin.

8. (canceled)

9. (canceled)

10. (canceled)

11. The pharmaceutical composition according to claim 1 wherein the first local anesthetic and the second local anesthetic are comprised in separate containers.

12. A method of prophylaxis of algophobia or odontophobia in a patient, comprising injecting the patient with a pharmaceutical composition comprising
a combination of a first local anesthetic and a second local anesthetic, wherein
the first local anesthetic is selected from the group consisting of amino amide-local anesthetics, and amino ester-local anesthetics, and pharmaceutically acceptable salts thereof, and
the second local anesthetic is prilocaine or a pharmaceutically acceptable salt thereof, and
optionally pharmaceutically acceptable excipients,
and wherein the first local anesthetic is not prilocaine, and
wherein
the first local anesthetic and the second local anesthetic are comprised in separate containers, and
wherein the first local anesthetic is administered before, simultaneously or after the second local anesthetic.

13. A method for treating pain in a patient comprising injecting a pharmaceutical composition comprising
a combination of a first local anesthetic and a second local anesthetic, wherein
the first local anesthetic is selected from the group consisting of amino amide-local anesthetics, amino ester-local anesthetics, and pharmaceutically acceptable salts thereof, and
the second local anesthetic is prilocaine or a pharmaceutically acceptable salt thereof, and
optionally pharmaceutically acceptable excipients,
and wherein the first local anesthetic is not prilocaine, and
wherein
the first local anesthetic and the second local anesthetic are comprised in separate containers, and
wherein the first local anesthetic is administered before,
simultaneously or after the second local anesthetic.

14. The method according to claim 12, wherein the first local anesthetic is lidocaine or a pharmaceutically acceptable salt thereof.

15. The method according to claim 12, wherein the first local anesthetic is administered before the second local anesthetic.

16. The method according to claim 12, wherein the patient previously has had inadequate local anesthesia, when in need thereof, when receiving one local anesthetic selected from the group consisting of amino amides and amino esters and pharmaceutically acceptable salts thereof.

17. The method according to claim 12, wherein the patient has at least one mutation in at least one allele of a MC1R gene, wherein the at least one mutation is a loss-of-function mutation.

18. (canceled)

19. (canceled)

20. (canceled)

21. The method according to claim 12, wherein the patient has phenotype red hair.

22. The method according to claim 13, wherein the first local anesthetic is lidocaine or a pharmaceutically acceptable salt thereof.

23. The method according to claim 13, wherein the first local anesthetic is administered before the second local anesthetic.

24. The method according to claim 13, wherein the patient previously has had inadequate local anesthesia, when in need thereof, when receiving one local anesthetic selected from the group consisting of amino amides and amino esters and pharmaceutically acceptable salts thereof.

25. The method according to claim 13, wherein the patient has at least one mutation in at least one allele of a MC1R gene, wherein the at least one mutation is a loss-of-function mutation.

26. The method according to claim 13, wherein the patient has phenotype red hair.
